(19) United States
(12) Patent Application Publication
(10) Pub. No.: US 2005/0096526 A1
(43) Pub. Date: May 5, 2005

(54) Endoscopy device comprising an endoscopy capsule or an endoscopy head with an image recording device, and imaging method for such an endoscopy device

(75) Inventor: Johannes Reinschke, Nurnberg (DE)

Correspondence Address: STAAS & HALSEY LLP, SUITE 700, 1201 NEW YORK AVENUE, N.W., WASHINGTON, DC, 20005, US

(73) Assignee: Siemens Aktiengesellschaft, Munich (DE)

(21) Appl. No.: 10/958,646

(22) Filed: Oct. 6, 2004

(30) Foreign Application Priority Data
Oct. 8, 2003 (DE) 10346678.9

(51) Int. Cl. A61B 5/05; A61B 6/00

(52) U.S. Cl. 600/407; 128/899; 600/476

(57) ABSTRACT

An endoscopy capsule or and endoscopy head has an image recording device for recording image from the interior of a hollow or vessel of the human or animal body. The capsule or head is rotatable. The optical axis of the image recording device is at an angle to the rotation axis during the rotation, making it possible, by digital reprocessing, to combine stroboscopically recorded individual images into a plane or relief-type, redundancy-free single image and to present (completely) an inner section of the hollow organ.

### CROSS REFERENCE TO RELATED APPLICATIONS

This application is based on and hereby claims priority to German Application No. 10346678.9 filed on Oct. 8, 2003, the contents of which are hereby incorporated by reference.

The present invention relates to an endoscopy device comprising an endoscopy capsule or an endoscopy head with an image recording device for recording images from the interior of a hollow organ or vessel of the human or animal body, which images can be transmitted wirelessly to an external receiver.

Besides known endoscopy in which an elongate endoscopy apparatus is to be pushed into the organ or vessel, a new procedure, capsule endoscopy, permits diagnosis of diseases of the gastrointestinal tract for example, especially of the upper sections of the small intestine (jejunum), the procedure permitting a painless examination, well tolerated by patients, of the entire area of the small intestine without radiation exposure; however, it is not restricted to this organ and instead can be used generally for examination of hollow organs or vessels. This examination procedure has the advantage that areas can be inspected in which known radiological and endoscopic procedures provide only inadequate diagnostic results.

In this procedure, the patient swallows a capsule provided with an image recording device, e.g. a miniature camera or miniature color video camera, which delivers a large number of individual images from the examined body area and permits painless, noninvasive diagnosis. If examination is to be carried out on an organ or vessel which is not accessible via the gastrointestinal tract, then the capsule is to be introduced into this vessel by another mechanism.

A capsule endoscope and a diagnostic system for viewing the entire mucosa of the small intestine is produced by the Israeli company Given Imaging Ltd. and sold under the name “M2A® Imaging Capsule”. This capsule is made up of a miniature color video camera, a light source, a miniature transmitter and an antenna. The housing of the capsule is made of a sealed biocompatible special material which is resistant to digestive secretions occurring in the gastrointestinal tract. The capsule is swallowed by the patient and is conveyed through the digestive tract by the peristaltic movement of the muscles of the stomach and intestines. The video capsule is ca. 11×26 mm in size, has a viewing field of approximately 140° and weights approximately four grams. It can be used to detect lesions measuring less than 0.1 mm. During a normal (eight-hour) examination procedure, the capsule generates approximately 57,000 images at a rate of two images per second. On completion of its passage through the digestive tract, the capsule is excreted naturally.

During the passage through the small intestine, the color video camera records image sequences which are sent in the form of ultrashort waves to a wireless receiving unit which is situated outside the body and which the patient wears on a belt around the waist, and, after demodulation, low-pass filtering and analog-to-digital conversion, are stored in a data recorder. The belt is comfortable to wear and, with the receiving device, allows the patient by and large to carry on with his or her everyday activities during the examination of the stomach and intestines.

Besides the use of capsule endoscopy in the area of the gastrointestinal tract, a great many other possible applications are presently being planned. As has been mentioned above, this generally involves endoscopic examination of hollow areas in the body in which the movement of the capsule is not impeded by the presence of connective tissue. This includes, for example, endovascular examination of the cerebral blood vessels, endoscopic examination of the bronchial tract (bronchoscopy), and minimally invasive endoscopic examination of the abdominal cavity and of the organs of the abdomen and pelvis (laparoscopy).

In the known endoscopy capsule described, the color video camera is oriented with its optical axis in alignment with the longitudinal axis of the capsule. This means the color video camera“looks” in the direction of the longitudinal axis either forward or rearward (depending on how the capsule has been received in, for example, the small intestine). As a result of this axial orientation of the optical axis of the video camera, the actual inner surface of the organ of interest is always recorded at an angle, despite the relatively large aperture angle of the camera. This can have the effect that very small lesions, or lesions lying in a depression or fold of the intestinal wall or the like, are not detected.

An alternative to this novel capsule endoscopy is known endoscopy in which an elongate endoscopy apparatus, with a forwardly situated endoscopy head containing the image recording device, is pushed into the hollow organ or vessel. In these endoscopy apparatus too, the optical axis of the image recording device, i.e. of the color video camera for example, is oriented in alignment with the longitudinal axis of the head, i.e. here too the camera looks forward in the direction of the longitudinal axis. The same problems thus also arise in the application of known endoscopy apparatus.

### SUMMARY OF THE INVENTION

The inventor focused on developing an endoscopy device which permits improved recording of the inner wall of organs or vessels for the purpose of improved, diagnostically relevant evaluation.

The inventor proposes an endoscopy device of the type mentioned at the outsethaving a mechanism provided at the capsule or head in order to permit a rotation of the capsule or of the endoscopy head, the optical axis of the image recording device being at an angle to the rotation axis during the rotation.

By virtue of the orientation of the optical axis of the image recording device, i.e. for example of the video camera, with respect to the rotation axis, which in the case of relatively narrow vessels or organs generally lies in the longitudinal axis of the vessel/organ, the inner wall is recorded in a kind of plan view. In contrast to the related art, the optical axis is not substantially parallel to the inner wall of the object to be recorded, but instead at an angle which is dependent on the degree of tilting. In connection with the rotation of the capsule or head as likewise provided for, it is now possible, after one complete rotation, to record a plan view of the inner wall of the vessel in the form of an annular section of defined length. The images recorded stroboscopically during a rotation are then digitized and combined into one image which represents the annular section in its entirety, i.e. without gaps, but without overlapping (that is to say redundancy-free). This makes it possible to detect even very small lesions which would not be able to be detected in an axially symmetrical orientation of the image recording device, so that an improved diagnostic evaluation is possible. Moreover, because of the substantially automatically controlled rotation of the capsule or of the endoscopy head in both examination procedures, the physician acquires a very rapid overview of the examination area.

According to a first embodiment, the optical axis can be at an angle to the longitudinal axis of the camera, which axis substantially coincides with the rotation axis. The camera is thus tilted in relation to the longitudinal axis of the capsule or head, and the rotation mechanism permits a rotation of the capsule or of the head about the longitudinal axis.

In an alternative embodiment, the optical axis is substantially in alignment with the longitudinal axis of the capsule or head, but the capsule or the head can be rotated about a rotation axis which is at an angle to its longitudinal axis. In this embodiment, the entire endoscopy capsule or endoscopy head is tilted in relation to the rotation axis, which in the final analysis offers the same effect as in the embodiment described above as far as the recorded images are concerned.

Various mechanisms can be used to permit a rotation of the capsule or head. In a first expedient embodiment, at least one permanent magnet which, for the rotation, interacts with an external, time-variable magnetic field. In an orientation of capsule or head where the optical axis is at an angle to the longitudinal axis of capsule or head and thus to the rotation axis, the permanent magnet is expediently arranged in such a way that its magnetization is substantially perpendicular to the longitudinal axis of the capsule or of the head, the external magnetic field rotating substantially perpendicular to the longitudinal axis of the capsule or of the head.

In the above-described embodiment in which the optical axis is in alignment with the longitudinal axis of the capsule or of the head and the entire capsule or head is tilted during the rotation, the permanent magnet is arranged in such a way that its magnetization is substantially parallel or perpendicular to the longitudinal axis of the capsule, the external magnetic field rotating at an angle of >0° and <90° (in parallel arrangement) or of >90° and <180° (in perpendicular arrangement) with respect to the rotation axis. The degree of tilt of the endoscopy capsule or of the head is dependent on the angle which the external magnetic field describes relative to the chosen rotation axis. Alternatively to this arrangement of the permanent magnet, the latter can also be arranged in such a way that its magnetization is at an angle of >0° and <90° to the longitudinal axis, the external magnetic field rotating substantially perpendicular to the rotation axis. In this case, the angle which the capsule or head describes during its rotation is dependent on the angle of tilt of the magnetization of the permanent magnet relative to the longitudinal axis of capsule or head.

As an alternative to the use of a magnet and of an external rotary magnetic field, the rotation mechanism provided at the capsule or head can also be designed as mechanical unit which, for the rotation, engage on the organ or vessel. The mechanical unit thus interacts directly with the inner wall of the hollow organ or vessel to be examined in order to bring about the rotation. It is conceivable to use grippers and the like which can be driven via an integrated miniaturized motor or the like. In the case of an endoscopy head, the rotation mechanism can also include an electric motor which is integrated in the head end, or in the device section adjacent thereto, and turns the head.

As has been described, the rotation affords the possibility of recording images of annular sections of the surface of the wall. A particularly advantageous embodiment involves the use of an optional additional mechanism for permitting a translatory movement of the capsule or of the head substantially in the direction of the rotation axis. With this mechanism it is possible, during imaging by rotation of the capsule or of the head, to move the endoscopy capsule or the head actively through the organ or vessel in the direction of the rotation axis, in order thereby to record the inner surface in quasi annular formation and without gaps along a relatively long distance (dependent on the particular organ/vessel being examined).

The translatory movement of the capsule or head or of the endoscopy apparatus can be achieved using an external, time-controlled magnetic gradient field. This can interact with the optionally already existing permanent magnet or with a permanent magnet specially provided for this purpose.

In a further alternative embodiment, the mechanism for the translatory movement, engages on the organ or vessel. This permits forward movement like that of a mole.

Finally, the additional mechanism can also be designed in the form of at least two electrodes provided on the outside of the capsule, via which electrodes an electrical stimulation impulse can be sent to the organ or vessel area surrounding the capsule in order to produce an area-limited contraction via which the capsule experiences a forward movement. By this electrical stimulation, it is possible to actively excite a local contraction of the vessel or organ, for example in the intestinal tract, so that the capsule, which in this case expediently narrows conically in the area of the stimulation electrodes, is moved forward. This alternative possibility of movement will primarily be used in the case of an endoscopy capsule, although a movement of a known endoscopy apparatus in this manner is also conceivable.

In a development of the concept, the endoscopy device moreover comprises an image processing unit which receives and processes the recorded images, transmitted from a suitable transmission device wirelessly (capsule) or by wire (endoscopy apparatus), and then outputs the images. The image processing unit used is advantageously designed to combine the individual images and to generate and output a flat image representation of the surface of the recorded organ or vessel on a monitor. As has been described, the rotation movement permits an annular recording or continuous annular recording (depending on the type of translatory movement). The image processing device is now able to suitably process the individual images and combine them, for example via suitable image analysis algorithms which permit superpositioning of the individual images based on corresponding image sections from two successively recorded images, etc. In addition, however, the image processing device is also able to treat the resulting image in such a way that the examination area which, as has been described, is scanned in an annular manner by virtue of the rotation, is represented as it were in a “sliced up” manner and as an “unrolled carpet”. Thus, a flat image representation is generated from the annular images. On account of the rotational camera movement and the translatory movement of the endoscopy head or capsule through the entire tubular organ or vessel (or the organ section of interest), the capsule or the endoscopy device delivers a complete, coherent, flat, redundancy-free image in the form of a top view of the inner wall of the organ or vessel.

The image processing device can be designed to generate a 2D image as a flat image representation or to generate a 3D image offering a relief-type view. In the case of the three-dimensional flat image representation, the observer is offered additional surface structure information which may be of advantage for the diagnostic evaluation.

A further embodiment is such that, for each image, the spatial position of the capsule or of the endoscopy head can be determined in a coordinate system of a position detection system, and the position data are used to determine the spatial position of the capsule/head in the organ or vessel of the examined body. This means that, for each individual image, a position detection is carried out so as to establish exactly where in the intestine or vessel, etc., the image was recorded. To do this, the capsule or head can, for example, emit a position-fixing signal in respect of its current position, and this signal can be located via suitable position detection sensors, which are positioned with respect to the patient, and the corresponding position data can be recorded. A particular advantage of recording the image position data relative to the body is that it is thereby possible to use a cursor or the like to select an image area of the flat image presented on the monitor, e.g. by clicking on the image or marking it with a window etc., and indicate to the physician the spatial position of the selected image area in the examined body. In this way, the physician is thus able to tell immediately the exact position of a recorded pathologically relevant irregularity such as a lesion or the like, and this is of advantage for planning a possibly required subsequent operation or treatment.

As has been described, the image recording device can be a video camera, in particular a color video camera. Alternatively, the images can be recorded by ultrasound, optical coherence tomography (OCT), fluorescence imaging or by other methods insofar as these imaging methods can be integrated in suitably miniaturized form into the capsule or into an endoscopy head.

In addition to the endoscopy device, the inventor prposes an imaging method for an endoscopy device comprising an endoscopy capsule or an endoscopy head equipped with an image recording device, the device being of the kind described above, and which imaging method comprises the following steps:

recording a sequence of individual images of the environment of the endoscopy capsule or endoscopy head which rotates and optionally executes a translatory movement, and transmitting the image data to a receiving and evaluating device of an image processing unit,

combining the individual images to generate a flat image representation showing the entire recorded area of the examined organ or vessel, and

outputting the flat image representation on a monitor.

The flat image representation which, as has been described, shows the tubular organ or the tubular vessel in as it were a sliced-up form or as an unwound coil, can be a 2D representation. Alternatively, it is also possible to generate a relief-type 3D representation.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 shows a diagrammatic sketch of an endoscopy device according to one embodiment of the invention,

FIG. 2 shows a diagrammatic representation of an endoscopy capsule in a first embodiment,

FIG. 3 shows a diagrammatic representation of an endoscopy capsule in a second embodiment,

FIG. 4 shows a diagrammatic representation of the processing of the individual images to generate the flat overall view, and

FIG. 5 shows a diagrammatic representation of an endoscopy device according to one embodiment of the invention, using a known endoscope.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

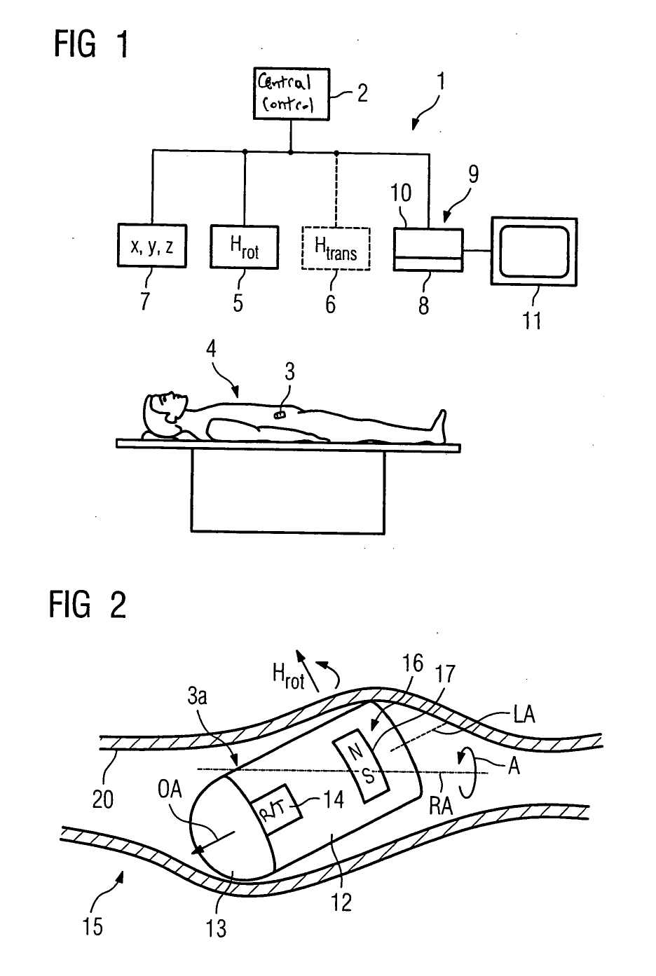

FIG. 1 shows a diagrammatic representation of a first embodiment of an endoscopy device 1 , comprising a central control device 2 which controls the operation of the relevant individual components. These individual components include on the one hand an endoscopy capsule 3 which, in the example shown, has been already introduced into the body of a patient 4. The patient 4 has for example swallowed the capsule, and the latter is located in the small intestine.

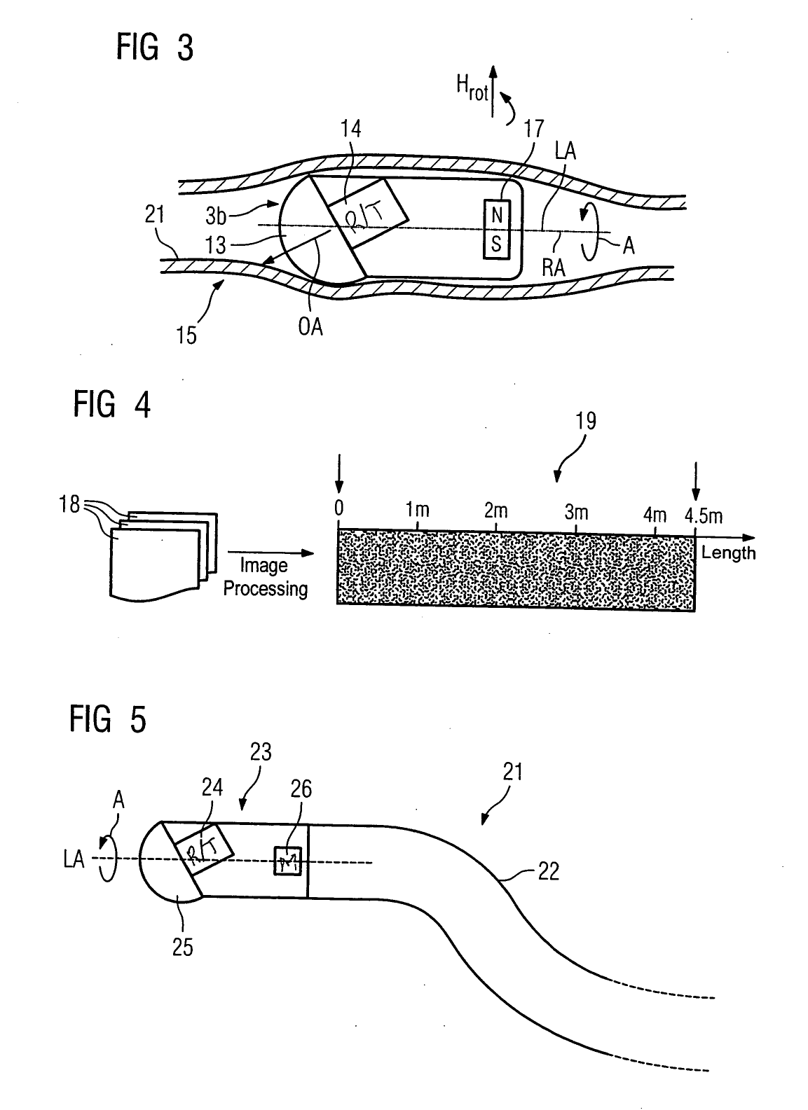

The endoscopy capsule 3 (which will be discussed in more detail below) can be actively rotated via an external magnetic field Hᵣₒₜ. For this purpose, a magnetic field generator 5 is provided which generates the time-variable, rotating magnetic field Hᵣₒₜ. A further magnetic field generator 6, shown in FIG. 1 by broken lines, can optionally be provided to effect a translatory movement of the endoscopy capsule 3 in the organ. This magnetic field generator 6 likewise generates a time-variable magnetic field in the form of a gradient magnetic field with separate field components in the x, y and z directions of a coordinate system. As has been stated, this magnetic field generator 6 is optional, and it is possible also to use other mechanisms to generate a translatory movement, as will be explained below with reference to FIGS. 2 and 3.

A position detection system 7 is also provided via which the position of the endoscopy capsule 3 can be detected in a coordinate system and thus in the body of the patient 4, so that it is known at all times at which organ position in the patient's body an image has been recorded by the endoscopy capsule 3.

As is known, the endoscopy capsule 3 is used to record images of the inner wall of the organ/vessel in which it is located. With a transmitter suitably provided at the capsule, the image data are transmitted to an external device 8 for receiving image data which is part of an image processing device 9. From the many individual images which are recorded and transmitted during the time when the endoscopy capsule 3 is located in the patient, the image processing module 10 of the image processing device 9 is finally able to generate an overall image which in the form of a flat image representation shows the recorded organ/vessel on a monitor 11 in a flat, sliced-up format. This too is discussed in more detail below.

FIG. 2 shows an enlarged view of an endoscopy capsule 3a in a first embodiment. As in capsules of this kind, this endoscopy capsule 3a comprises a capsule housing 12 made of a biocompatible material. At one end, a window 13 is provided, which is adjoined downstream by an image recording device with an integrated or assigned image transmission device, hereinafter only referred to as image recording and transmitting device (R/T) 14, e.g. a color video camera with a video transmitter, and which records the environment via the window 13. A suitable transmitter (not shown) is used for wireless transmission of the image data to the receiving device 8 for further processing.

In the endoscopy capsule 3a, the optical axis OA of the image recording and transmitting device 14 is axially symmetrical with respect to the longitudinal axis LA of the endoscopy capsule 3a. To permit an annular imaging of the inner wall 20 of a tubular organ, for example the small intestine 15, a mechanism 16 is provided in the inside of the endoscopy capsule 3a to permit a rotation of the endoscopy capsule 3a with at the same time the possibility of tilting the endoscopy capsule 3a relative to the rotation axis RA. The rotation axis RA coincides substantially with the longitudinal axis of the section of the tubular organ in which the capsule is momentarily situated. In the present case, the mechanism 16 is designed as a permanent magnet 17 whose magnetization, indicated by the two poles N and S, is substantially perpendicular to the longitudinal axis LA of the endoscopy capsule. To generate a rotation and also a tilting of the endoscopy capsule 3a relative to the pre-defined rotation axis RA, use is made of the external magnetic field Hᵣₒₜ which, in the example shown, is likewise located and rotates at an angle to the rotation axis, by which mechanism the rotation axis is defined. On account of the magnetic coupling, the permanent magnet 17 orients itself according to the external field Hᵣₒₜ, which on the one hand leads to a tilting of the longitudinal axis of the endoscopy capsule 3a relative to the defined rotation axis, and, on the other hand, by virtue of the magnetic field rotation, to the desired capsule rotation itself.

Because of the tilting of the entire endoscopy capsule 3a, the optical axis of the image recording and transmitting device 14 is at an angle to the rotation axis and thus at an angle to the inner wall 20, so that the latter can be recorded in the manner of a plan view. The annular scanning of the entire inner wall is effected by the rotation. In this embodiment, there is on the whole a rotary/gyratory capsule movement brought about by the magnetic field rotation and the capsule design.

There is also the possibility, as has already been mentioned with respect to FIG. 1, of using an additional magnetic field generator 6 to generate a translatory magnetic field which serves to move the endoscopy capsule 3a in the direction of the rotation axis, actively controlled by the organ. Alternatively to this, another mechanism to convey the capsule can be used, as are described below in FIG. 3.

FIG. 3 shows a further embodiment of an endoscopy capsule 3b. In terms of its structure, this corresponds substantially to the endoscopy capsule 3a, but here the image recording and transmitting device 14 is from the outset tilted relative to the longitudinal axis LA of the capsule in alignment with the rotation axis RA. For this purpose, the window 13 is already arranged on a correspondingly inclined capsule housing section, and the image recording and transmitting device 14 is positioned following the tilt of the window. Here too, the optical axis OA is at an angle to the longitudinal axis LA and to the rotation axis RA.

Here too, means provided for effecting the mechanism for rotation is a permanent magnet 17 which likewise interacts with an external magnetic field Hᵣₒₜ to bring about the rotation. Here too, the permanent magnet is arranged with its magnetization, indicated by the two magnetic poles N and S, perpendicular with respect to the longitudinal axis LA of the capsule. However, it is not necessary here for the magnetic field to be rotated at an angle or tilt, since in this case the endoscopy capsule is not to be tilted itself, since the optical axis OA is at an angle to the longitudinal axis LA. Instead, the external magnetic field Hᵣₒₜ can in this case likewise rotate substantially perpendicular to the longitudinal axis LA of the capsule, as is shown in FIG. 3.

Since, in this case too, rotation is effected and the optical axis OA is at an angle to the inner wall 20 of the organ 15, images of the wall can be recorded in the form of plan views.

If a magnetic field is used to obtain the translatory movement, it is conceivable to use this magnetic field to move the capsule as it were intermittently along a defined path Δx, to execute a complete rotation so that a complete annular section has been recorded, and then to execute a further intermittent movement by a path increment Δx so that a multiplicity of individual annular section sequences can be recorded and then processed.

The possibility of obtaining an electrically stimulated movement of the capsule, by using at least two electrodes arranged on the outside of the housing, is not shown here. The wall section of the organ or vessel near the electrodes is impacted by a current impulse via these electrodes, which leads to the contraction of this area, as a result of which the capsule is pushed forward section by section. In this case, the capsule is designed narrowing conically in the area remote from the window.

FIG. 4 shows, finally, a diagrammatic representation of the image processing and an example of a generated image. The figure shows a plurality of individual images 18 which have been recorded by the image recording and transmitting device 14. In the image processing module 10, these images are now combined using suitable image analysis and image processing algorithms and in such a way, based on the image sections which correspond in two successive images and can be recorded by suitable analysis algorithms, that an overall picture is obtained which presents the whole of the scanned inner wall of the organ/vessel, specifically in a sliced up or uncoiled, flat format, as is shown in the form of the representation 19 in FIG. 4. This image shows, for example along a length of ca. 4.5 m, the inner surface of the small intestine in the form of a flat surface representation, generated on the basis of the individual images of the annular or spiral scanning of the wall. Since the organ position and the corresponding position data have preferably been recorded via the position detection system 7 for each individual image, it is possible to assign defined image sections of the representation 19 to defined organ positions. FIG. 4 shows an example of an axis (length) which in the illustrative embodiment shown is the axial coordinate of the tubular organ, and which makes it possible to rapidly record sections at distinct points (in the example shown the pylorus (at 0 m) and the ileocecal valve (at 4.5 m) in the gastrointestinal tract). The physician can thus very quickly pinpoint where a specific irregularity in the organ has occurred.

It goes without saying that the monitor 11 does not have to display the entire image representation 19 showing the inner wall along a length of 4.5 m in the example shown. Instead, it is possible for the physician to view the overall image in sections or, using a suitable scroll bar, to move the image or enlarge or reduce sections, etc.

Finally, it should also be noted that it is also possible, by selecting a defined image section or area of the image representation 19, to automatically indicate the associated position data of the image section in the patient's body or in the recorded organ, which is expedient for preparing for a subsequent operation or the like, and equally also for the diagnosis itself.

Finally, FIG. 5 shows a further embodiment, of an endoscopy device in which, in contrast to the above-described embodiments, use is made of a known endoscopy apparatus 21 composed of an elongate, wire-like or tube-like portion 22 and of the endoscopy head 23 which, in this case too, has an image recording and transmitting device 24 positioned at an angle to the longitudinal axis LA of the endoscopy head 23, comparable to the embodiment according to FIG. 3. Images of the area around the head can thus be recorded via an inclined window 25 provided at the front end.

In contrast to the capsule designs, the image recording and transmitting device RT 24 in this case does not communicate wirelessly with the external image processing device, and instead this is done by a cable link (not indicated here). The signal cables through which the image data are routed to the outside are routed through the wire-like or tube-like portion 22.

In this embodiment too, the endoscopy head 23 is rotatable relative to the stationary tubular or wire-like portion 22, for which purpose it is appropriately mounted on the latter. The rotation is obtained in this case by a miniaturized electric motor 26 integrated in the endoscopy head 23 in the example shown here. It is equally possible, however, for this electric motor 26 to be positioned near the head at the end of the portion 22. The electric motor 26 can also be powered and controlled via lines routed through the portion 22.

Instead of the electric motor 26, it is of course also possible here to integrate a permanent magnet which interacts with an external rotary magnetic field in order to rotate the endoscopy head relative to the tubular or wire-like portion 22.

Although it is possible in this case, because of its length, to push the endoscopy apparatus 21 through the organ/vessel from the outside to its target, it is equally possible, of course, to provide a corresponding mechanism for automatic translatory movement. For example, a permanent magnet (not shown) can be integrated in the endoscopy head 23 and interact with an external gradient magnetic field. All the other way of obtaining the translatory movement which have been described in the above illustrative embodiments can also conceivable be used.

The invention has been described in detail with particular reference to preferred embodiment thereof and examples, but it will understood that variations and modifications can be effected within the spirit and scope of the invention covered by the claims which may include the phrase “at least one of A, B and C” or a similar phrase as an alternative expression that means one or more of A, B and C may be used, contrary to the holding in Superguide v. DIRECTV, 69 USPQ2d 1865 (Fed. Cir. 2004)

1. An endoscopy device comprising:
an endoscopy capsule or head;
an image recording device provided in the capsule or head, to record images along an optical axis of the image recording device, the image recording device recording images of a hollow organ or vessel of an animal, from an interior of the hollow organ or vessel; and
a rotation device provided at the capsule or head to rotate the capsule or head about a rotation axis, the optical axis of the image recording device being at an angle to the rotation axis.

2. The endoscopy device as claimed in claim 1, wherein
the capsule or head extends about a longitudinal axis thereof,
the optical axis is at an angle to the longitudinal axis of the capsule or head, and
the longitudinal axis substantially coincides with the rotation axis.

3. The endoscopy device as claimed in claim 1, wherein
the capsule or head extends about a longitudinal axis thereof,
the optical axis is substantially in alignment with the longitudinal axis of the capsule or head, and
the rotation axis is at an angle to the longitudinal axis.

4. The endoscopy device as claimed in claim 1, wherein the rotation device comprises at least one permanent magnet which interacts with an external, time-variable magnetic field to rotate the capsule or head.

5. The endoscopy device as claimed in claim 4, wherein
the capsule or head extends about a longitudinal axis thereof,
the permanent magnet is positioned such that its magnetization is substantially perpendicular to the longitudinal axis of the capsule or head, and
the external, time-variable magnetic field rotates substantially perpendicular to the longitudinal axis of the capsule or head.

6. The endoscopy device as claimed in claim 4, wherein
the capsule or head extends about a longitudinal axis thereof,
the permanent magnet is positioned such that its magnetization is parallel to the longitudinal axis of the capsule or head, and
the external, time-variable magnetic field rotates at an angle between 0° and 90° with respect to the rotation axis.

7. The endoscopy device as claimed in claim 4, wherein
the capsule or head extends about a longitudinal axis thereof,
the permanent magnet is positioned such that its magnetization is perpendicular to the longitudinal axis of the capsule or head, and
the external, time-variable magnetic field rotates at an angle between 90° and 180° with respect to the rotation axis.

8. The endoscopy device as claimed in claim 4, wherein
the capsule or head extends about a longitudinal axis thereof,
the permanent magnet is positioned such that its magnetization is at an angle between 0° and 90° with respect to the longitudinal axis, and
the external, time-variable magnetic field rotages substantially perpendicular to the rotation axis.

9. The endoscopy device as claimed in claim 1, wherein the rotation device engages on the organ or vessel.

10. The endoscopy device as claimed in claim 1, wherein
the endoscopy device comprises an endoscopy head, and
the rotation device comprises an integrated electric motor.

11. The endoscopy device as claimed in claim 1, further comprising a movement device to permit a translatory movement of the capsule head substantially in a direction of the rotation axis.

12. The endoscopy device as claimed in claim 11, wherein the movement device comprises a permanent magnet which, for the translatory movement, interacts with an external, time-variable translational magnetic field.

13. The endoscopy device as claimed in claim 12, wherein the rotation device comprises an external, time-variable rotational magnetic field, which interacts with the permanent magnet.

14. The endoscopy device as claimed in claim 12, wherein
the capsule or head extends about a longitudinal axis thereof,
permanent magnet is positioned such that its magnetization is substantially in alignment with the longitudinal axis, and
the translational magnetic field is a gradient field.

15. The endoscopy device as claimed in claim 11, wherein the movement device engages with the organ or vessel.

16. The endoscopy device as claimed in claim 11, wherein the movement device comprises:
at least two electrodes provided on the outside of the capsule or head; and
an impulse generator to send an electrical stimulation impulse to the organ area or vessel, via the electrodes, at an area surrounding the capsule or head in order to produce an area-limited contraction via which the capsule or head experiences a forward movement.

17. The endoscopy device as claimed in claim 1, further comprising an image processing unit to receive and process recorded images from the image recording device to combine individual images and to generate and output on a monitor a flat image representation of a surface of the organ or vessel.

18. The endoscopy device as claimed in claim 17, wherein the image processing unit generates a 2D image.

19. The endoscopy device as claimed in claim 17, wherein the image processing unit generates a a 3D relief image.

20. The endoscopy device as claimed in claim 17, wherein, for each individual image, a spatial position of the capsule or head is determined in a coordinate system, and the spatial position is used to determine a position of the capsule or head in the organ or vessel of the examined body.

21. The endoscopy device as claimed in claim 20, wherein a cursor is used to select an image area of the flat image representation output on the monitor, so that the spatial position of the selected image area is determined and output.

22. The endoscopy device as claimed in claim 1, wherein the image recording device is a video camera selected from the group consisting of a color picture video camera, an ultrasonic imager, an OCT imager and a fluorescence imager.

23. An imaging method for an endoscopy device, comprising:
recording a sequence of individual images from a rotating endoscopy capsule or head positioned within a hollow organ or vessel of an animal, the images being captured along an optical axis by an image recording device which is rotating about a rotation axis, the optical axis being at an angle to the rotation axis;
transmitting image data from the individual images to a receiving and evaluating device;
combining the individual images to generate a flat image representation showing an entire recorded area of the organ or vessel; and
outputting the flat image representation on a monitor.

24. The imaging method as claimed in claim 20, wherein the flat image representation is generated and output as a 2D representation.

25. The imaging method as claimed in claim 20, wherein the flat image representation is generated and output as a relief-type 3D representation.

26. The endoscopy device as claimed in claim 1, wherein the animal is a human.

27. The imaging method as claimed in claim 23, wherein the animal is a human.
